(19) United States
(12) Patent Application Publication
(10) Pub. No.: US 2026/0027561 A1
(43) Pub. Date: Jan. 29, 2026

(54) DEVICES AND METHODS ENABLING CELLS TO UNDERGO BOTH VISUAL AND MOLECULAR DIAGNOSTICS

(71) Applicant: Corramedical, Inc., San Diego, CA (US)

(72) Inventors: Wilfrido MOJICA, Amherst, NY (US); Alexander ARROW, Lakeside, CA (US)

(21) Appl. No.: 18/782,937

(22) Filed: Jul. 24, 2024

(51) Int. Cl. B01L 3/00; G01N 33/543

(52) U.S. Cl. CPC B01L 3/502715; B01L 3/502753; B01L 3/502761; G01N 33/54326; B01L 2200/025; B01L 2200/0652; B01L 2300/0816; B01L 2300/0864; B01L 2400/0406

(57) ABSTRACT

Devices and methods that enable loose cells to undergo both visual and molecular diagnostics are disclosed herein. In an embodiment, a microfluidic chip includes an input area, an output area, a microchannel, and a viewing area. The input area includes an input silo that extends from an upper surface. The output area includes an output silo configured to extend from a lower surface. The microchannel places the input silo and the output silo in fluid communication, such that the cells can flow from the input silo, through the microchannel, to the output silo to be output for the molecular diagnostic. The viewing area is in fluid communication with the microchannel and is configured to enable the visual diagnostic. In an embodiment, a supportive device includes a mount for the microfluidic chip and an alignment arm that translates a magnet into and out of alignment with the viewing area.

### BACKGROUND

### Technical Field

The present disclosure generally relates to devices and methods that enable cells to undergo both visual and molecular diagnostics. More specifically, the present disclosure generally relates to devices and methods enabling a fine needle aspirate sample, or loose cells generated by “touch prep” following a core needle biopsy, to be analyzed visually on a microfluidic chip and then outputted from the microfluidic chip for a molecular diagnostic process.

### Background Information

Solid tumor diagnostic procedures sometimes involve a solid tissue biopsy, most often obtained through a hollow needle approximately 1 mm wide, a procedure known as a core needle biopsy (CNB). Other times they involve a less invasive procedure for harvesting cells from a target tissue of interest by way of a thin needle only wide enough to obtain loose cells, without regard for obtaining a solid piece of tissue, called a fine needle aspiration (FNA). FNA procedures have become commonplace in cancer diagnostic workups, and are portrayed to patients as simple procedures with low risk of adverse events. A specialized syringe is used, and the advantage for the patient is that this is a less invasive way of obtaining a pathology diagnosis. In the current standard of care, the extracted cells are placed on a glass slide and remain there for visual examination, known as a cytology examination. Cells from the FNA procedure are smeared on to a glass slide, stained and in some cases, cover-slipped. FIGS. 1A to 1C illustrate this process, showing cells harvested from the FNA procedure placed on a standard glass slide, smeared between two sides, and then covered with a coverslip and sent for microscopic examination. The cells can then be examined under a microscope. In an alternate situation in which loose cells on a glass slide are evaluated visually (via microscopy), some clinicians who perform core needle biopsies will take the solid tissue core harvested and, before subjecting it to the preservative formalin, gently touch the tissue to a glass slide. This so-called “touch prep” procedure leaves cells on the glass slide for staining and microscopic examination.

These existing methods do not provide any information about the genes (the DNA) of the patient's cells. If the oncologist treating the patient desires any molecular studies (e.g. genomic screening of next generation sequencing) to be performed on the patient's cells, the only way to accomplish that is to (a) subject the patient to a second FNA procedure to harvest more cells, or (b) a tedious, expensive, and error-prone process to gain access to the cells fixed under the glass slide and selectively capture them, wherein a user needs to immerse the glass slide in an organic solvent like xylene (dangerous) to loosen the coverslip, remove it, then scrape the cells into another tube (potential source of losing cells) or use a laser capture microdissection device (expensive) for molecular testing.

### SUMMARY

Diagnostic testing modalities have expanded in recent years to include an increased number of tests to identify molecular changes. Visual examination of cells on a glass slide under a microscope remains one of the most useful (and time-honored) diagnostic techniques in medicine, and comprises the basis for the field of pathology. The present disclosure provides devices and methods that enable both of visual and diagnostic modalities from a single FNA procedure. As a secondary function, devices and methods disclosed herein also provide the user with a simple way to know that the FNA specimen contains cells (the intent of a FNA procedure) immediately upon withdrawing the specimen from the patient. That information has the advantages that it (a) helps avoid unnecessary additional needle passes (if the first specimen contains adequate cells), and (b) helps avoid ending a procedure without obtaining tumor cells (if the first specimen does not contain adequate cells). The devices and methods of the present disclosure thus provide a dual purpose: (1) confirming “specimen adequacy” or “representative diagnosis” through examination on the chip, and quick recovery of those same cells or their nucleic acids for (2) molecular testing.

No similar product currently exists on the market. Instruments to sort cells exist, but they do not allow cytology-quality examination. An advantage of using the devices and methods of the present disclosure is that cells harvested in a FNA procedure (or “touch prep” following a CNB procedure) that would otherwise be entombed in a glass slide are instead immediately available for molecular testing after they have been examined via microscopy. In some instances, when molecular studies are requested, the cells from an FNA are the only place where there is enough material to test. Recovering these cells can be an expensive process that can take days, as it involves removing the glued-on coverslip and may require laser capture microdissection.

The embodiments described herein can be used after an FNA procedure, or a “touch prep” subsequent to a CNB procedure, is performed. The embodiments provide a convenient simple-to use device that takes a fine needle aspirate or “touch prep” sample and (1) confirms “specimen adequacy” or “representative diagnosis” through visual examination, and (2) enables quick recovery of those same cells or their nucleic acids for molecular testing, then also (3) retains and routes those cells for their status quo usage-preservation of their cytologic image for microscopic examination. In an embodiment, the disclosed devices and methods capture cells using immunomagnetic beads, cause the cells to travel through a microfluidic channel to an area for viewing or scanning, then enable recovery of the cells for molecular analysis. The embodiments described herein are applicable and apply equally well to either: (a) loose cells on a glass slide produced by a FNA procedure, or (b) loose cells on a glass slide produced by a “touch prep” subsequent to a CNB.

The disclosed devices and methods can be used in a clinical environment, for example, in instances where a pathologist is called into a radiology procedure (fine needle aspiration, or core needle biopsy) and is being asked to assess (1) adequacy and (2) confirmation that diagnostic material is present (tumor cells). More specifically, the presently disclosed devices and methods provide microfluidic chip systems and technologies for interventional radiologists and other clinicians who perform fine needle aspiration (FNA) procedures and the pathologists who assist them with diagnostic methodologies to (a) ascertain whether an FNA procedure has yielded adequate cellular material, and to (b) segregate cells in a fluid suspension of the harvested cells into one or more aliquots for microscopic examination with recovery of those same cells for molecular or genomic testing. The microfluidic chip can be about the size of a standard glass slide for a microscope, with a linear channel that originates at a vertical cellular input silo, runs through a widened viewing area, and terminates in an upward/downward facing cellular output silo, along with a specialized supporting device that moves a magnet into and out of alignment with the widened viewing area. The purpose of the device is that cells harvested in a FNA procedure, or by way of a “touch prep” following a CNB procedure, that would otherwise be entombed in a glass slide and unavailable for molecular testing are instead available for molecular testing after they have been examined via microscopy.

A first aspect of the present disclosure is to provide a microfluidic chip enabling cells to undergo both a visual diagnostic and a molecular diagnostic. The microfluidic chip includes a body, an input area, an output area, at least one microchannel, and a viewing area. The body includes an upper surface and a lower surface extending in a longitudinal direction from a first end to a second end and in a lateral direction from a first lateral side to a second lateral side. The input area includes an input silo configured to receive cells obtained from a patient. The input silo extends from the upper surface at the first end of the body. The output area includes an output silo configured to output the cells received at the input silo. The output silo is configured to extend from the lower surface at the second end of the body. The design aligns the input silo and the output silo in fluid communication, such that the cells can flow from the input silo, through the at least one microchannel, to the output silo to be output for the molecular diagnostic. The viewing area is in fluid communication with the at least one microchannel and is configured to enable the visual diagnostic of the cells that have flowed through the at least one microchannel.

A second aspect of the present disclosure is to provide a system including the microfluidic chip and a supportive device configured to serve as a platform from which the microfluidic chip can be mounted and aligned to a magnetic force with the viewing area.

A third aspect of the present disclosure is to provide a supportive device enabling cells to undergo both a visual diagnostic and a molecular diagnostic. The supportive device includes a microfluidic chip mount and an alignment arm. The microfluidic chip mount is configured to removably receive a microfluidic chip in an orientation in which cells can be deposited at an input area of the microfluidic chip and flow through the microfluidic chip via capillary action. The alignment arm includes a magnet and is configured to move with respect to the microfluidic chip mount to translate the magnet into and out of alignment with a viewing area of the microfluidic chip when the microfluidic chip is mounted on the microfluidic chip mount.

A fourth aspect of the present disclosure is to provide a system including the supportive device and the microfluidic chip having the input area and the viewing area.

A fifth aspect of the present disclosure is to provide a method enabling cells to undergo both a visual diagnostic and a molecular diagnostic. The method includes depositing cells attached to immunomagnetic beads into an input area in fluid communication with at least one microchannel such that the cells attached to the immunomagnetic beads flow from the input area through the at least one microchannel, aligning a magnetic force with a viewing area in fluid communication with the at least one microchannel so that the cells attached to the immunomagnetic beads collect in the viewing area for the visual diagnostic, and enabling the cells attached to the immunomagnetic beads to flow to an output area in fluid communication with the viewing area to be collected for the molecular diagnostic.

A sixth aspect of the present disclosure is to provide a system including the microfluidic chip and the supportive device described herein.

A seventh aspect of the present disclosure is to provide a method of using the microfluidic chip and the supportive device described herein.

Other objects, features, aspects and advantages of the apparatuses and methods disclosed herein will become apparent to those skilled in the art from the following detailed description, which, taken in conjunction with the annexed drawings, discloses exemplary embodiments of the disclosed apparatuses and methods.

### BRIEF DESCRIPTION OF THE DRAWINGS

Referring now to the attached drawings which form a part of this original disclosure:

FIGS. 1A to 1C illustrate an example embodiment of a prior art method of smearing cells harvested from an FNA procedure on a glass slide for analysis;

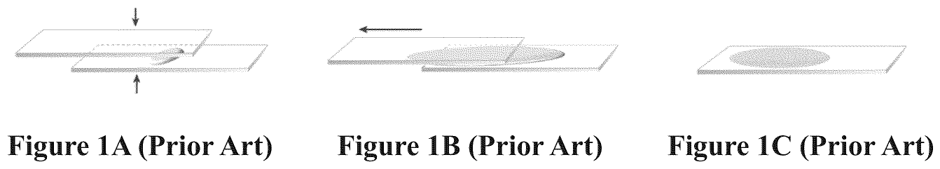

FIG. 2 illustrates a top perspective view of an example embodiment of a prototype microfluidic chip in accordance with the present disclosure.

FIG. 3 illustrates a top perspective view of an example embodiment of a microfluidic chip in a first orientation in accordance with the present disclosure;

FIG. 4 illustrates a bottom perspective view of the microfluidic chip of FIG. 3 in the first orientation;

FIG. 5 illustrates a top perspective view of the microfluidic chip of FIGS. 3 and 4 in a second orientation in accordance with the present disclosure.

FIG. 6 illustrates a top perspective diagram of the microfluidic chip of FIGS. 3 to 5 changing orientation in accordance with the present disclosure;

FIG. 7 illustrates a top perspective view of an example embodiment of a mechanical attachment mechanism for a microfluidic chip in accordance with the present disclosure;

FIG. 8 illustrates a top perspective view of an example embodiment of a supportive device in accordance with the present disclosure;

FIG. 9 illustrates a top perspective view of the microfluidic chip of FIG. 2 mounted on the supportive device of FIG. 8; and

FIG. 10 illustrates an example embodiment of a method of using a microfluidic chip and/or a supportive device in accordance with the present disclosure.

### DETAILED DESCRIPTION OF EMBODIMENTS

Selected embodiments will now be explained with reference to the drawings. It will be apparent to those skilled in the art from this disclosure that the following descriptions of the embodiments are provided for illustration only and not for the purpose of limiting the invention as defined by the appended claims and their equivalents.

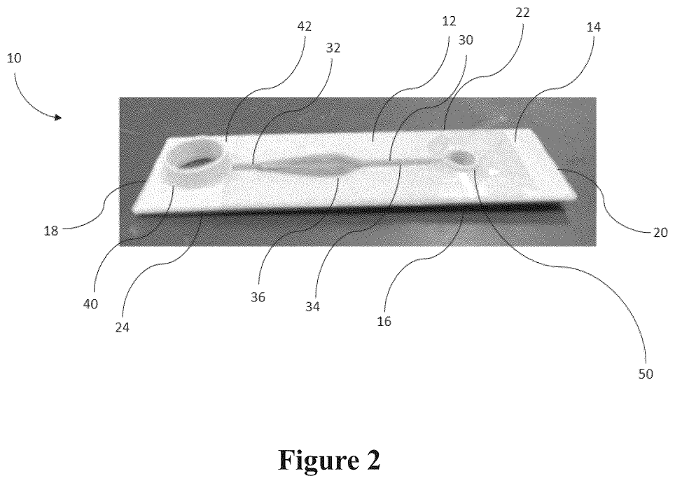

FIGS. 2 to 6 illustrate an example embodiment of a microfluidic chip 10 enabling cells to undergo both a visual diagnostic and a molecular diagnostic in accordance with the present disclosure. The microfluidic chip 10 is configured to maximize the clinical benefits of limited amounts of cellular material obtained by a FNA procedure or “touch prep” following a CNB procedure. In use, the microfluidic chip 10 enables cells attached to magnetic beads to move through a microchannel, stop at a certain point, be examined by a microscope (or digitally scanned), then move to the end of the microfluidic chip 10 and be captured for molecular testing.

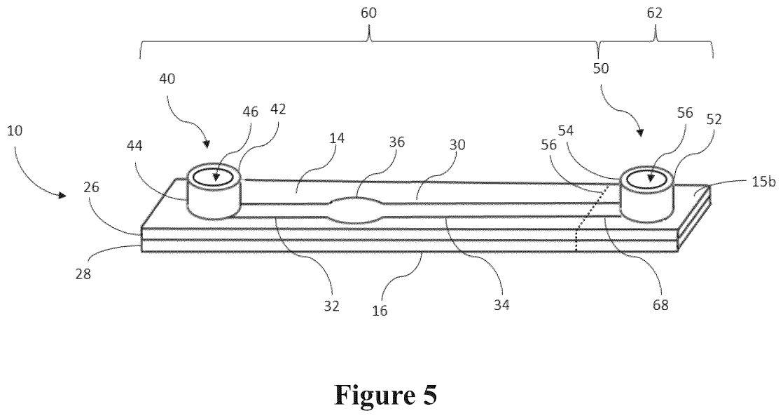

As seen in FIGS. 2 to 6, the microfluidic chip 10 includes an elongated body 12 having an upper surface 14 and a lower surface 16. The upper surface 14 and the lower surface 16 extend longitudinally from a first end 18 to a second end 20, and extend laterally from a first lateral side 22 to a second lateral side 24. The body 12 can be formed, for example, of glass, silicon or polymer. In an embodiment, the body 12 is formed by a first (or upper) body 26 and a second (or lower) body 28 which are attached together to form the body 12 such that the first body 26 forms the upper surface 14 and the second body 28 forms the lower surface 16. Alternatively, the body 12 can be formed from a single piece of material (e.g., a single piece of glass, silicon or polymer). In the illustrated embodiment, the body 12 is approximately the size of a standard glass slide for a microscope. As discussed in more detail below, the body 12 is adjustable from a first orientation O1 (shown in FIGS. 2 to 4) to a second orientation O2 (shown in FIG. 5).

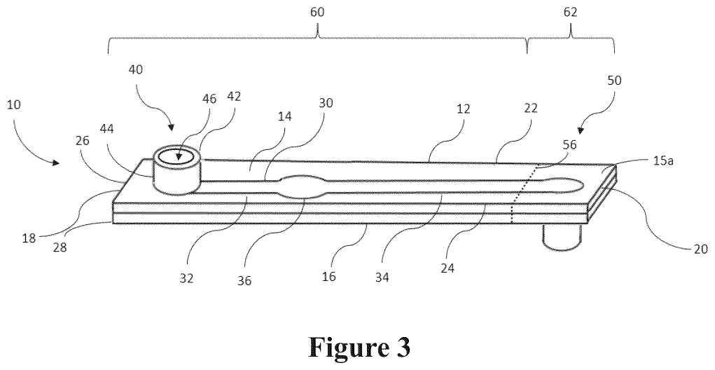

The microfluidic chip 10 includes at least one microchannel 30. In the illustrated embodiment, the at least one microchannel 30 includes a first microchannel 32 and a second microchannel 34. The microfluidic chip 10 also includes a viewing area 36. The viewing area 36 is in fluid communication with the at least one microchannel 30. As discussed in more detail below, the viewing area 36 is configured to enable a visual diagnostic of the cells that have flowed through the at least one microchannel 30. As seen in FIGS. 2 and 3, the first microchannel 32 and the second microchannel 34 extend in the longitudinal direction of the body 12. The viewing area 36 is located between the first microchannel 32 and the second microchannel 34 in the longitudinal direction. The first microchannel 32 and the second microchannel 34 are longer than the viewing area 36 in the longitudinal direction. The viewing area 36 is wider than the first microchannel 32 and the second microchannel 34 in the lateral direction. The first microchannel 32 and the second microchannel 34 are each in fluid communication with the viewing area 36, such that fluid with cells can travel through the first microchannel 32 to the viewing area 36, and then travel from the viewing area 36 through the second microchannel 34.

The at least one microchannel 30 can be embedded, etched or molded into the body 12. In an embodiment, the at least one microchannel 30 is embedded, etched or molded into the upper surface 14 of the body 12. In an embodiment, the at least one microchannel 30 is embedded, etched or molded into the first body 26 or the second body 28 before the first body 26 is attached to the second body 28. In an embodiment, the at least one microchannel 30 is positioned within the body 12 between the first surface 14 and the second surface 16. In an embodiment, the at least one microchannel 30 is formed between the first body 26 and the second body 28 when the first body 26 and the second body 28 are attached together in a leak tight manner.

As seen in FIGS. 2 and 3, the microfluidic chip 10 includes an input area 40. The input area 40 includes a first (or input) silo 42. The first silo 42 is configured to receive cells obtained from a patient so that the cells can undergo the visual diagnostic. The first silo 42 extends from the upper surface 14 at the first end 18 of the body 12. More specifically, the first silo 42 includes an outer wall 44 that extends outward from the upper surface 14 of the body 12. The input silo 42 includes an inner area 46 located within the outer surface 44. The inner area 46 is in fluid communication with the first microchannel 32. In use, a user (e.g., clinician or interventional radiologist) can place a needle or pipette containing cells from an FNA procedure into the input silo 42 and deposit the cells, so that the cells can flow through the first microchannel 32 to the viewing area 36. More specifically, the user can deposit fluid with immunomagnetically bound cells into the input silo 42.

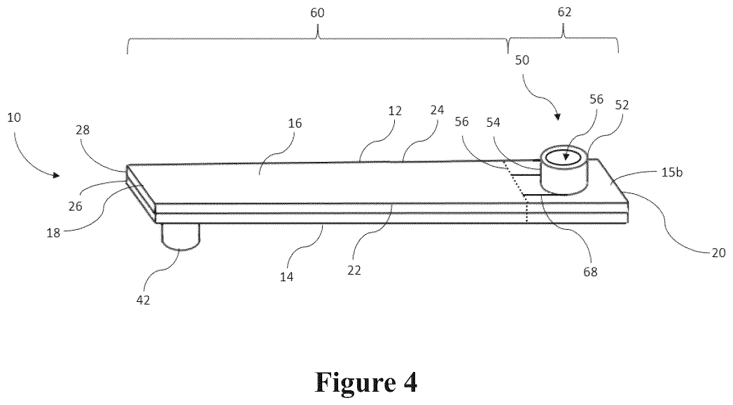

As seen in FIG. 4, the microfluidic chip 10 includes an output area 50. The output area 50 includes a second (or output) silo 52. The second silo 52 is configured to output the cells received at the input silo 42 so that the cells can undergo the molecular diagnostic. The second silo 52 is configured to extend from the lower surface 16 at the second end 20 of the body 12. More specifically, the second silo 52 includes an outer surface 54 that is configured to extend outward from the lower surface 16 of the body 12. The output silo 52 includes an inner area 56 located within the outer surface 54. The inner area 56 is in fluid communication with the second microchannel 34. In use, fluid containing cells from an FNA procedure can flow from the viewing area 36 through the second microchannel 32 to the output silo 52 to be collected for a molecular diagnostic. In the orientation shown in FIG. 4, the output silo 52 is in a downward position, which enables cells and fluid to flow through microchannel(s) 30, 32, 34 to the output silo 52 via capillary action. The at least one microchannel 30 places the first silo 42 and the second silo 52 in fluid communication, such that the cells can flow from the first silo 42, through the at least one microchannel 30, to the second silo 52 to be output for a molecular diagnostic.

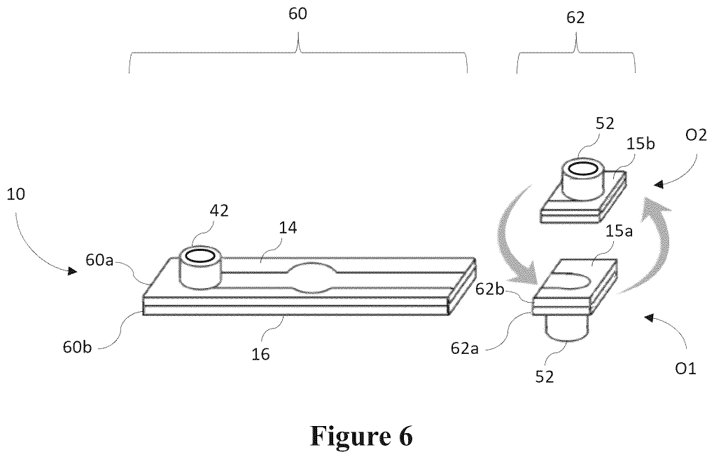

In the illustrated embodiment, the microfluidic chip 10 includes a first (or inlet) part 60 and a second (or outlet) part 62. The first part 60 includes the input area 50, and the second part 62 includes the output area 50. In FIGS. 3 and 4, a boundary line 56 marks the location where the second part 62 is configured to separate and/or move with respect to the first part 60. By separating and/or moving the second part 62 with respect to the first part 60, the microfluidic chip 10 can change between a first (or downward) orientation O1 and a second (or upward) orientation O2. FIGS. 3 and 4 illustrate the microfluidic chip 10 in the first (downward) orientation O1, FIG. 5 illustrates the microfluidic chip 10 in the second (upward) orientation O2, and FIG. 6 illustrates how the second part 62 can separate from and/or move with respect to the first part 60 so that the microfluidic chip 10 changes between the first and second orientations O1, O2. As illustrated, the second part 62 is configured to move with respect to the first part 60 so that the output silo 52 extends from the upper surface 14 in a same direction as the input silo 42. In an embodiment, the second part 62 separates partially but not completely from the first part 60 when the microfluidic chip 10 changes from the first orientation O1 to the second orientation O2, or vice versa.

As seen in FIGS. 3 and 4, when the microfluidic chip 10 is in the first orientation O1, the first silo 42 projects upward from the upper surface 14, and the second silo 52 projects downward from the lower surface 16. Thus, the input silo 42 and the output silo 52 extend from the body 12 in opposite directions in the first orientation O1. In this orientation, a first surface 15a of the second part 62 is part of the upper surface 14, and a second surface 15b of the second part 62 is part of the lower surface 16. As seen in FIG. 4, the second silo 52 extends outward from the second side 15b of the second part 62.

As seen in FIG. 5, when the microfluidic chip 10 is in the second orientation O2, both the first silo 42 and the second silo 52 project upward from the upper surface 14. Thus, the input silo 42 and the output silo 52 extend from the body 12 in a same direction in the second orientation O2. In this orientation, the first surface 15a of the second part 62 becomes part of the lower surface 16, and the second surface 15b of the second part 62 becomes part of the upper surface 14. To change to the second orientation O2, as seen in FIG. 6, the second part 62 rotates and/or flips to change the vertical orientation of the second silo 52 between the first (downward) orientation O1 and the second (upward) orientation O2.

When the second (output) silo 52 is in the first (downward) orientation O1, the microfluidic chip 10 allows cells to flow through the first microchannel 32 until they reach the central widened viewing area 36. With the second silo 52 in the first orientation O1, gravity causes fluid to flow along the pathway of the first microchannel 32 and through the cells therein via capillary action. Each drop of fluid that leaves the microfluidic chip 10 pulls another drop of fluid volume to take its place, hence the movement of fluid. By contrast, when the second silo 52 in the second (upward) orientation O2, the fluid remains stationary, so cells will remain in central viewing area 36 while on a microscope stage or scanning micrograph area. When the user translates a magnet to be positioned directly underneath the widened viewing central area 36 as discussed herein, as the fluid and immunomagnetically bound cells pass over the magnet, the bound cells thereby become stationary.

As seen in FIGS. 4 and 5, in an embodiment, the second part 62 includes a partial microchannel 68 that is placed in fluid communication with the at least one microchannel 30 when the second part 62 moves so that the output silo 52 extends from the upper surface 14 in the same direction as the input silo 42. More specifically, the second part 62 includes a partial microchannel 68 that is placed in fluid communication with the first microchannel 32, the second microchannel 34 and/or the viewing area 36 when the microfluidic chip 10 is in the second orientation O2 (FIG. 5).

In an embodiment, the microfluidic chip 10 can be formed and dimensioned such that either of the first silo 42 or the second silo 52 can be used as an input silo, with the other of the first silo 42 and the second silo 52 being used as an output silo. For example, both the input area 40 and the output area 42 can be made so as to separate and/or move with respect to a central part of the body 12 including the viewing area 36. This embodiment prevents the user from mistakenly pipetting cells and fluid into the wrong silo 42, 52 because either silo 42, 52 can be used as the input silo or output silo.

In an embodiment, the microfluidic chip 10 can be formed from a plurality of pieces. More specifically, each of the first part 60 and the second part 62 can be formed from a plurality of pieces. As seen in FIG. 6, the first part 60 can be formed of a first piece 60a and a second piece 60b. The first piece 60a can include at least part of the at least one microchannel 30 (e.g., in FIG. 6, the first microchannel 30 and part of the second microchannel 34), the viewing area 36 and/or the first silo 42. The second piece 60b includes a flat base. The second part 62 can also be formed of a first piece 62a and a second piece 62b. The first piece 62a can include at least part of the at least one microchannel 30 (e.g., in FIG. 6, part of the second microchannel 34) and/or the second silo 52. The second piece 60b includes a flat base. The first part 60 can be formed by attaching the first piece 60a to the second piece 60b, and the second part 62 can be formed by attaching the first piece 62a to the second piece 62b. In an embodiment, the first piece 60a attaches to the second piece 60b using a snap-fit, and the first piece 62a attaches to the second piece 62b using a snap-fit. In an embodiment, the at least one microchannel 30 (including the first microchannel 32 and/or the second microchannel 34) and/or the viewing area 36 can be formed between the first piece 60a and the second piece 60b when the first piece 60a is attached to the second piece 60b. Similarly, part of the at least one microchannel 30 (e.g., another part of the second microchannel 34) can be formed between the first piece 62a and the second piece 62b when the first piece 62a is attached to the second piece 62b.

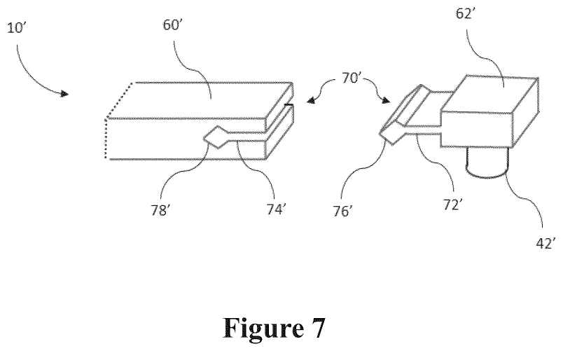

FIG. 7 illustrates an example embodiment of a microfluidic chip 10′ with a mechanical attachment mechanism 70′ configure to enable the second part 62 including the second silo 42′ to separate from the first part 60′. The microfluidic chip 10′ can include all of the elements of the microfluidic chip 10 illustrated in FIGS. 2 to 6. In the illustrated embodiment of FIG. 7, one of the first part 60′ and the second part 62′ includes a projection 72′ and the other of the first part 60′ and the second part 62′ includes a corresponding groove 74′. An outer part 76′ of the projection 72′ forms a key with a unique shape (e.g., here, a diamond), and an inner part 78′ of the groove 74′ has the same unique shape so that the projection 72′ can slide laterally into the groove 74′ with the second part 62′ in either the first orientation O1 or the second orientation O2. Those of ordinary skill in the art will recognize from this disclosure that there are other ways of removably attaching the first part 60, 60′ and the second part 62, 62′ that enable adjustment between the first orientation O1 and the second orientation O2.

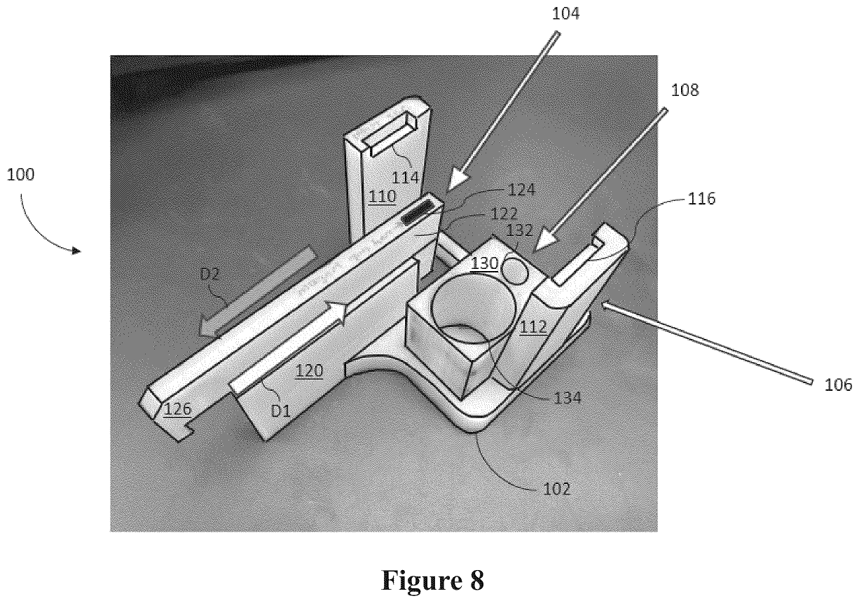

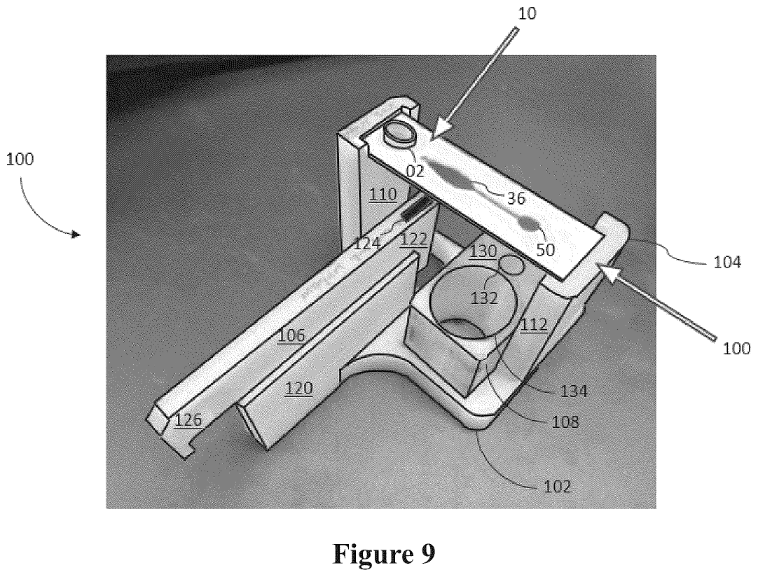

FIGS. 8 and 9 illustrate an example embodiment of a supportive device 100 configured to be used in combination with the microfluidic chip 10. FIG. 8 illustrates the supportive device 100 without the microfluidic chip 10 mounted thereon, while FIG. 9 illustrates the microfluidic chip 10 mounted on the supportive device 100. As discussed in more detail below, the supportive device 100 enables cells from an FNA procedure to undergo both a visual diagnostic and a molecular diagnostic.

In the embodiment illustrated in FIGS. 8 and 9, the supportive device 100 includes a base 102, a microfluidic chip mount 104, an alignment arm 106 and an output container 108. In the illustrated embodiment, the microfluidic chip mount 104, the alignment arm 106 and the output container 108 are attached to each other via the base 102. In an embodiment, the supporting device 100 can be formed by 3D printing or injection molding.

The microfluidic chip mount 104 is configured to removably receive a microfluidic chip 10 in an orientation in which cells can be deposited at an input area 40 of the microfluidic chip 10 and flow through the microfluidic chip 10 via capillary action. The microfluidic chip mount 104 includes a first side mount 110 and a second side mount 112 configured to hold the microfluidic chip 10 in place while the alignment arm 106 is moved into and out of alignment with the microfluidic chip 10. As seen in FIG. 8, the first side mount 110 includes a first indentation 114 configured to receive one end of the microfluidic chip 10, and the second side mount 112 includes a second indentation 116 configured to receive the other side of the microfluid chip 10. FIG. 9 illustrates the microfluidic chip 10 mounted with one end in the first indentation 114 and the other end in the second indentation 116.

The alignment arm 106 is configured to translate a magnetic force into and out of alignment with the microfluidic chip 10. More specifically, the alignment arm 106 includes a magnet 124 and is configured to move with respect to the microfluidic chip mount 104 to translate the magnet 124 into and out of alignment with the viewing area 36 of the microfluidic chip 10 when the microfluidic chip 10 is mounted on the microfluidic chip mount 104. In the illustrated embodiment, the alignment arm 106 moves with respect to a stationary part 120 of the base 102. The alignment arm 106 has a first end 122 including the magnet 124 and a second end 126 configured to be gripped by a user. In the illustrated embodiment, the magnet 124 is fitted into an aperture in the first end 122. When the user grips the second end 126 and causes the alignment arm 106 to travel in the first direction D1, the alignment arm 106 translates the magnet 124 into alignment with the viewing area 36 of the microfluidic chip 10. When the user causes the alignment arm 106 to travel in the opposite second direction D2, the alignment arm 106 translates the magnet 124 out of alignment with the viewing area 36 of the microfluidic chip 10. In the illustrated embodiment, the magnet 124 is aligned with the viewing area 36 of the microfluidic chip 10 when it is located vertically below the viewing area 36. Thus, in an embodiment, the alignment arm 106 is configured to translate the magnet 124 vertically beneath the viewing area 36 of the microfluidic chip 10 to place the magnet 124 into alignment with the viewing area 36.

In the illustrated embodiment, the alignment arm 106 moves linearly in the lateral direction of the microfluidic chip 10. In other embodiments, the alignment arm 106 can move in other ways and/or directions, for example, can be rotated or translated vertically or diagonally with respect to the microfluidic chip 10. In another alternative embodiment, the alignment arm 106 and/or its magnet 124 can remain stationary and the microfluidic chip mount 104 can move the microfluidic chip 10 into alignment with the magnet 124.

The output container 108 is positioned vertically beneath the output area 50 of the microfluidic chip 10 when the microfluidic chip 10 is mounted on the microfluidic chip mount 104. More specifically, the output container 108 is positioned vertically beneath the second silo 52 when the microfluidic chip 10 is mounted by the microfluidic chip holder 104 with the second silo in the first (downward) orientation O1. In the illustrated embodiment, the output container 108 includes a fluid collecting area 130 that is located beneath the second silo 52 when the microfluidic chip 10 is mounted by the microfluidic chip holder 104. The fluid collecting area 130 can include a first opening 132 to collect fluid that drips out of the second silo 52 when the second silo 52 is in the first (downward) orientation O1. In an embodiment, the first opening 132 removably receives a container configured to collect the fluid that drips out of the second silo 52 when the second silo 52 is in the first orientation O1. In the illustrated embodiment, the output container 108 also includes a second opening 134 to collect excess fluid as the cells move in the at least one microchannel 30, 32, 34 until aligned with the magnet 124 and ready for viewing. In an embodiment, the second opening 134 removably receives a container configured to collect the fluid that drips out of the second silo 52 when the second silo 52 is in the first orientation O1. In an embodiment, the second (larger) opening is used to collect fluid that helps move cells to the viewing area 36, and then the first (smaller) opening is used to collect the cells. In an embodiment, the output container 108 can move with respect to the microfluidic chip holder 104 (or vice versa) such that the output area 130 moves into and out of alignment with the second silo 52. More specifically, the output container 108 can move with respect to the microfluidic chip holder 104 (or vice versa) such that either of the first opening 132 or the second opening 134 can alternatively be located underneath the second silo 52.

In the illustrated embodiment, the movements of the microfluidic chip 10 and the supportive device 100 are manual. It will be understood by those of ordinary skill in the art from this disclosure that one or more operations of the microfluidic chip 10 and/or the supportive device 100 can be made automatic. For example, in an embodiment, the supportive device 100 can be include one or more motor and/or processor configured to automatically introduce cells into the first silo 42, translate the moving arm 106 into and/or out of alignment with the microfluidic chip 10, translate the output container into and/or out of alignment with the microfluidic chip 10, record images of cells while located in the viewing area 36 of the microfluidic chip 10, and/or move the second part 62 between the first (downward) orientation O1 and the second (upward) orientation O2.

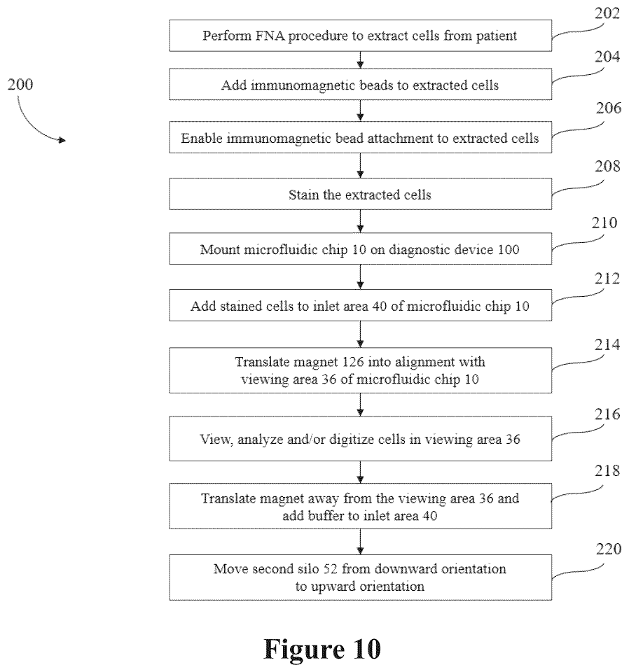

FIG. 10 illustrates an example embodiment of a method 200 of using the microfluidic slide 10 and/or the supportive device 100 in accordance with the present disclosure. In an embodiment, the method 200 is a method of preparing and routing cells for both a visual diagnostic and a molecular diagnostic. Those of ordinary skill in the art will recognize from this disclosure that certain steps of the method 200 can be added, removed or altered without departing from the spirit and scope of the present disclosure. Those of ordinary skill in the art will also recognize from this disclosure that certain steps of the method 200 can be used with other microfluidic chips 10 and/or supportive devices 100 besides those disclosed herein disclosed herein.

At step 202, a fine needle aspiration (FNA) procedure is performed to extract cells from a patient. The FNA procedure can be performed using a specifically designed needle that is placed by an interventional radiologist into a suspected tumor mass in an organ, such as the liver, breast, lung, kidney, etc. The extracted cells can be placed in a container such as a microcentrifuge tube.

At step 204, immunomagnetic beads are added to the extracted cells. The immunomagnetic beads are magnetic beads with an antibody to a cell membrane protein attached. The immunomagnetic beads can be added to the container (e.g., microcentrifuge tube) already containing the cells. The amount of immunomagnetic beads to add to the container may vary and can be determined though reasonable experimentation.

At step 206, the immunomagnetic beads attach to the extracted cells. More specifically, the extracted cells and immunomagnetic beads are incubated to cause the immunomagnetic beads to attach to the extracted cells. The extracted cells and immunomagnetic beads can be incubated by placing the container (e.g., microcentrifuge tube) containing the extracted cells and immunomagnetic beads into an incubator. The extracted cells and immunomagnetic beads typically only need to be incubated for a few minutes for the immunomagnetic beads to attach to the extracted cells. The incubation time may vary. In one embodiment, the extracted cells and immunomagnetic beads can be incubated for approximately 5 minutes. In another embodiment, they can be incubated for approximately 20 minutes.

At step 208, the extracted cells with immunomagnetic beads attached are stained. In an embodiment, the extracted cells are stained with a supravital stain. One such stain is methylene blue works well, but those of ordinary skill in the art will recognize from this disclosure that other stains can also be used. The extracted cells can be stained while still in the container (e.g., microcentrifuge tube).

At step 210, the microfluidic slide 10 is mounted on the supportive device 100. In the embodiment shown in FIGS. 8 and 9, the microfluidic slide 10 is placed into the supportive device 100 with one end in the first indentation 114 and the other end in the second indentation 116. Those of ordinary skill in the art will recognize from this disclosure that there are other ways to sufficiently mount the microfluidic slide 10 on the supportive device 100. At this point, the microfluidic slide 10 is in the first (downward) orientation O1.

At step 212, the user adds the stained cells to the input area 40 of the microfluidic chip 10. In the illustrated embodiment, the user pipettes the stained cells with immunomagnetic beads attached into the input silo 42 while the microfluidic slide 10 is mounted on the supportive device 100. The stained cells will move through the first microchannel 32 to the viewing area 36 due to capillary action as discussed above. In an embodiment, the user pipettes fluid into the microfluidic chip 10 before pipetting the fluid with the stained cells. This is to ensure that the microchannel 30, 32, 34 is already filled with fluid and to prevent the addition of bubbles

At step 214, the magnet 124 is aligned with the viewing area 36 of the microfluidic chip 10. More specifically, the user causes the alignment arm 106 to move the magnet 124 into alignment with the viewing area 36. In the illustrated embodiment, the user causes the alignment arm 106 to move the magnet 124 into alignment with the viewing area 36 by translating the magnet 124 linearly in the lateral direction of the microfluidic chip 10 until the magnet 124 is located vertically beneath the viewing area 36. The magnet 124 serves to align and enrich for the cells of interest (bound to the immunomagnetic beads) when the magnet 124 is located below the viewing area 36. When the magnetic force is aligned with the viewing area 36, the cells attached to the immunomagnetic beads collect in the viewing area 36 to enable a visual diagnostic.

At step 216, once the cells are aligned in the viewing area 36, the visual diagnostic can be performed. In the illustrated embodiment, the microfluidic chip 10 can be taken off of the microfluidic chip mount 104 and viewed under a conventional bright field microscope, and/or images of the cells in the viewing area 36 can be digitized using a scanner. In another embodiment, the supportive device 100 includes a scanner configured to digitize images of the fluid in the viewing area 36 while the magnet 124 is located beneath the viewing area 36. In another embodiment, a visual diagnostic can be performed while the microfluidic chip 10 is mounted on the microfluidic chip mount 104.

At step 218, once the cells have been viewed and characterized, the user can enable the cells attached to the immunomagnetic beads to flow to the output area 50 to be collected for a molecular diagnostic. In an embodiment, the user can cause the magnet 124 to translate in the second direction D2 out of alignment, place the microfluidic chip 10 back on the microfluidic chip holder 104, and add buffer fluid through the input silo 42 so as to initiate movement of the cells through the second microchannel 34 towards the output silo 52, where the cells can be collected for downstream testing like molecular analysis.

At step 220, the microfluidic chip 10 can be lifted out of the chip mount 104, and the second part 62 can be moved from the first (downward) orientation O1 to the second (upward) orientation O2 as discussed herein. With the second part 62 and thus the second silo 52 in the second orientation O2, there is no more fluid flow and the microfluidic chip 10 can be placed under a microscope for examination of the cells (or scanning).

The embodiments described herein provide improved devices and methods for both visual and molecular diagnostics of cells. A key advantage of the method enabled by the microfluidic chip 10 and/or the supportive device 100 is that there is maximal utilization of scarce diagnostic cells for purposes of cytologic diagnosis and molecular evaluation in a time efficient, cost-effective manner. It should be understood that various changes and modifications to the devices and methods described herein will be apparent to those skilled in the art and can be made without diminishing the intended advantages.

### GENERAL INTERPRETATION OF TERMS

In understanding the scope of the present invention, the term “comprising” and its derivatives, as used herein, are intended to be open-ended terms that specify the presence of the stated features, elements, components, groups, and/or steps, but do not exclude the presence of other unstated features, elements, components, groups, integers and/or steps. The foregoing also applies to words having similar meanings such as the terms, “including”, “having” and their derivatives. Also, the terms “part,” “section,” or “element” when used in the singular can have the dual meaning of a single part or a plurality of parts.

The term “configured” as used herein to describe a component, section or part of a device includes hardware and/or software that is constructed to carry out the desired function.

While only selected embodiments have been chosen to illustrate the present invention, it will be apparent to those skilled in the art from this disclosure that various changes and modifications can be made herein without departing from the scope of the invention as defined in the appended claims. For example, the size, shape, location or orientation of the various components can be changed as needed and/or desired. Components that are shown directly connected or contacting each other can have intermediate structures disposed between them. The functions of one element can be performed by two, and vice versa. The structures and functions of one embodiment can be adopted in another embodiment. It is not necessary for all advantages to be present in a particular embodiment at the same time. Every feature which is unique from the prior art, alone or in combination with other features, also should be considered a separate description of further inventions by the applicant, including the structural and/or functional concepts embodied by such features. Thus, the foregoing descriptions of the embodiments according to the present invention are provided for illustration only, and not for the purpose of limiting the invention as defined by the appended claims and their equivalents.

What is claimed is:

1. A microfluidic chip enabling cells to undergo both a visual diagnostic and a molecular diagnostic, the microfluidic chip comprising:
a body including an upper surface and a lower surface extending in a longitudinal direction from a first end to a second end and in a lateral direction from a first lateral side to a second lateral side;
an input area including an input silo configured to receive cells obtained from a patient, the input silo extending from the upper surface at the first end of the body;
an output area including an output silo configured to output the cells received at the input silo, the output silo configured to extend from the lower surface at the second end of the body;
at least one microchannel placing the input silo and the output silo in fluid communication, such that the cells can flow from the input silo, through the at least one microchannel, to the output silo to be output for the molecular diagnostic; and
a viewing area in fluid communication with the at least one microchannel and configured to enable the visual diagnostic of the cells that have flowed through the at least one microchannel.

2. The microfluidic chip of claim 1, wherein
the body is adjustable from a first orientation to a second orientation,
the input silo and the output silo extend from the body in opposite directions in the first orientation, and
the input silo and the output silo extend from the body in a same direction in the second orientation.

3. The microfluidic chip of claim 1, wherein
the body includes a first part and a second part,
the first part includes the input area,
the second part includes the output area, and
the second part is configured to move with respect to the first part so that the output silo extends from the upper surface in a same direction as the input silo.

4. The microfluidic chip of claim 3, wherein
the second part includes a partial microchannel that is placed in fluid communication with the at least one microchannel when the second part moves so that the output silo extends from the upper surface in the same direction as the input silo.

5. The microfluidic chip of claim 1, wherein
the at least one microchannel includes a first microchannel and a second microchannel, and
the viewing area is located between the first microchannel and the second microchannel in the longitudinal direction.

6. The microfluidic chip of claim 5, wherein
the viewing area is wider than the first microchannel and the second microchannel in the lateral direction.

7. A system comprising the microfluidic chip of claim 1 and a supportive device configured to mount the microfluidic chip and align a magnetic force with the viewing area.

8. A supportive device enabling cells to undergo both a visual diagnostic and a molecular diagnostic, the supportive device comprising
a microfluidic chip mount configured to removably receive a microfluidic chip in an orientation in which cells can be deposited at an input area of the microfluidic chip and flow through the microfluidic chip via capillary action; and
an alignment arm including a magnet, the alignment arm configured to move with respect to the microfluidic chip mount to translate the magnet into and out of alignment with a viewing area of the microfluidic chip when the microfluidic chip is mounted on the microfluidic chip mount.

9. The supportive device of claim 8, wherein
the alignment arm is configured to translate the magnet vertically beneath the viewing area of the microfluidic chip to place the magnet into alignment with the viewing area.

10. The supportive device of claim 8, comprising
an output container positioned vertically beneath an output area of the microfluidic chip when the microfluidic chip is mounted on the microfluidic chip mount.

11. The supportive device of claim 10, comprising
a base attaching the microfluidic chip mount, the alignment arm and the output container.

12. The supportive device of claim 8, wherein
the alignment arm includes a first end and a second end,
the first end includes the magnet, and
the second end is configured to be gripped by a user to cause the alignment arm to translate the magnet into and out of alignment with the viewing area of the microfluidic chip.

13. The supportive device of claim 8, wherein
the microfluidic chip mount includes a first side mount and a second side mount,
the first side mount includes a first indentation configured to receive one end of the microfluidic chip, and
the second side mount includes a second indentation configured to receive an opposite end of the microfluid chip.

14. A system comprising the supportive device of claim 8 and the microfluidic chip having the input area and the viewing area.

15. A method enabling cells to undergo both a visual diagnostic and a molecular diagnostic, the method comprising:
depositing cells attached to immunomagnetic beads into an input area in fluid communication with at least one microchannel such that the cells attached to the immunomagnetic beads flow from the input area through the at least one microchannel;
aligning a magnetic force with a viewing area in fluid communication with the at least one microchannel so that the cells attached to the immunomagnetic beads collect in the viewing area for the visual diagnostic; and
enabling the cells attached to the immunomagnetic beads to flow to an output area in fluid communication with the viewing area to be collected for the molecular diagnostic.

16. The method of claim 15, comprising
extracting the cells from a patient during a fine needle aspiration procedure.

17. The method of claim 15, wherein
aligning the magnetic force with the viewing area includes translating a magnet into a location beneath the viewing area.

18. The method of claim 15, wherein
enabling the cells attached to the immunomagnetic beads to flow to the output area includes translating the magnetic force away from the viewing area.

19. The method of claim 15, comprising
mounting a microchip including the input area, the at least one viewing area and the microchannel onto a supportive device prior to depositing the cells attached to the immunomagnetic beads into the input area.

20. The method of claim 19, comprising
adjusting an orientation of the microfluidic chip after the visual diagnostic to prevent the cells attached to magnetic beads from further flowing out of the output area via capillary action.
